(19) United States
(12) Patent Application Publication
(10) Pub. No.: US 2008/0317807 A1
(43) Pub. Date: Dec. 25, 2008

(54) STRONTIUM FORTIFIED CALCIUM NANO-AND MICROPARTICLE COMPOSITIONS AND METHODS OF MAKING AND USING THEREOF

(75) Inventors: Weijia William Lu, Hong Kong (HK); Zhaoyang Li, Pokfulam (HK); Wing Moon Raymond Lam, Yuen (HK); Dip Kei Keith Luk, Pokfulam (HK)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: The University of Hong Kong

(21) Appl. No.: 12/143,076

(22) Filed: Jun. 20, 2008

Related U.S. Application Data
(60) Provisional application No. 60/945,680, filed on Jun. 22, 2007.

(51) Int. Cl. A61K 9/00; A61K 9/14; A61K 9/20; A61K 9/48; A61P 19/08; A61K 33/06; A61K 33/42

(52) U.S. Cl. 424/422; 424/489; 424/464; 424/451; 424/682; 424/602

(57) ABSTRACT

Compositions containing strontium fortified calcium nanoparticles and/or microparticles, and methods of making and using thereof are described herein. The strontium fortified calcium compounds contain calcium ions, calcium atoms, strontium ions, strontium atoms, and combinations thereof and one or more anions. Exemplary anions include, but are not limited to, citrate, phosphate, carbonate, and combinations thereof. The particles can be formulated for enteral or parenteral administration by incorporating the particles into a pharmaceutically carrier. The compositions can further contain one or more active agents useful for bone diseases or disorders, such as vitamin D, growth factors, and combinations thereof. The compositions can be used to treat or prevent one or more bone diseases or disorders of the bone, such as osteoporosis. Alternatively, the particles can be coated onto a substrate, such as the surface of an implant. The coatings can be used to improved biocompatibility of the implant, prevent loosening of the implant, reducing leaching of metal ions from metallic implants, and reduce corrosion. The coatings can be applied to the substrate using a variety of techniques well known in the art. In one embodiment, the coating is applied using electrophoretic deposition. The use of nano- and/or microparticles that provide high surface area helps to improve interfacial strength between the coating and the implant, which allows for the use of lower sintering temperatures. Lowering sintering temperatures minimizes or prevents thermal decomposition of the coating material and/or degradation of the implant material.

### CROSS-REFERENCE TO RELATED APPLICATIONS

This application claims priority to U.S. Ser. No. 60/945,680 entitled “Nano and Micro Strontium Fortified Calcium Compounds for Implant Surface Coating and Treatment/Prophylaxis of Osteoporosis and Bone Diseases” filed Jun. 22, 2007. The disclosures in the application listed above are herein incorporated by reference.

### FIELD OF THE INVENTION

The present application relates to strontium fortified calcium nano- and/or microparticle compositions for use as implant coatings, fillers, and scaffold materials, as well as pharmaceutical compositions for the treatment of. osteoporosis and other bone diseases and disorders.

### BACKGROUND OF THE INVENTION

As the population ages, osteoporosis continues to become more prevalent. Osteoporosis can dramatically increase fracture risk by reducing bone tensile strength and compressive strength. Bone remodeling imbalance is the major cause of osteoporosis. Throughout life, old bone is continuously removed by bone-resorbing osteoclasts and replaced with new bone which is formed by osteoblasts in a highly regulated manner. In aging or pathological conditions, bone resorption outpaces new bone formation, which results in osteoporosis.

Vitamin D plus calcium has been administered for the treatment of osteoporosis. While this formulation is safe, it is has been shown to be ineffective in reversing bone loss. Calcium and vitamin D supplements have been administered in combination with other anabolic or anti-resorptive agents for osteoporosis treatment. However, these treatments have also been relatively ineffective. In osteoporosis patients, stand alone calcium supplement treatment does not significantly improve bone mineral density. While high serum calcium concentrations have been shown to suppress resorption (which explains why calcium supplements can reduce bone loss in osteoporosis patient), calcium does not induce formation of osteoblasts for synthesizing new matrix material, and thus no new bone will form despite adequate serum calcium levels.

Strontium salts have been shown to enhance bone formation and retard bone resorption. In vitro strontium increases mature osteoblast collagen and non-collagenic proteins synthesis. Strontium concentrations of 10⁻³M or higher have been shown to increase collagen and non-collagenic protein synthesis by 34%. Strontium salts have a large therapeutic range (e.g. 10-2000 mg) and exhibit and low toxicity.

Strontium divalent cations (typically in the form of a strontium salts) have been shown to inhibit bone resorption by direct and/or matrix-mediated inhibition of osteoclast activity and differentiation. Inhibition of osteoclast activity and differentiation accounts for the anti-resorptive properties of strontium. In an osteoporotic mice model induced by parathyroid hormone, strontium ranelate inhibited bone resorption. It increased DNA synthesis by three- to four-fold in fibroblast and pre-osteoblastic enriched cell populations, and also enhanced pre-osteoblastic cell replication.

Strontium ions can increase alkaline phosphatase activities in both male (53%) and female rats (56%). Significant increase of plasma IGF-1 was also observed in males after 104 weeks of treatment but not in females. IGF-1 is known to increase synthesis in osteoblasts, stimulate bone matrix apposition and collagen synthesis and bone growth. Strontium has been shown to increase serum calcium concentrations in a rat model while decreasing total protein concentration.

Strontium-calcium compositions have been investigated. U.S. Pat. No. 5,442,536 to Aoki, et al. discloses implant materials that are produced by coating a core material with a calcium phosphate type compound and converting the coating layer into an apatite type ceramic layer via a hydrothermal treatment. The disclosed options for apatite-type layers are calcium phosphate, strontium apatite, magnesium apatite, chlorine apatite, fluorine apatite and carbonate apatite.

U.S. Pat. No. 6,905,723 to Li discloses a method of preparing an implant with a strontium-substituted ceramic apatite coating. The method involves incubating the surface of the implant with a composition comprising strontium ions, calcium ions, phosphate ions and a liquid carrier.

U.S. Pat. No. 6,338,810 to Carpena, et al. discloses a process for manufacturing apatite ceramic for biological use. The '810 patent discloses that including strontium in the apatite ceramic structure is useful because it facilitates bone regeneration.

U.S. Patent Application No. 2002/0127711 to Kale, et al. discloses the ex vivo formation of mammalian bone and subsequent uses for this bone. Kale describes the method of producing bone by obtaining an osteogenic or bone precursor cell; culturing the cell in the presence of osteogenic growth factors and establishing the cells cultures such that the bone is formed within the cells of the bone cell spheroid that results. In one embodiment cells are introduced to the body via a non-biological matrix, which may contain strontium fortified calcium hydroxyapatite.

U.S. Patent Application No. 2008/0027455 to Boudeville, et al. discloses an injectable cement comprising a mineral solid phase (containing strontium fortified calcium compositions), a liquid phase, and optional polymer microparticles. The mineral solid phase is composed of a mixture of powders having the molar composition (CP)₆(CsO)_{y}(SrCO₃)_{z}. The compositions contain calcium hydrogenphosphate dehydrate (DCPD), anhydrous calcium hydrogenphosphate (DCPA), anhydrous mixed calcium strontium hydrogen phosphate, an equimolar mixture of calcium bis(dihydrogenphosphate monohydrate (MCPM) and calcium oxide in a mixture of two or three of these compounds.

PCT Application WO 01/49327 to Versitech, Ltd. (“Versitech”) discloses bone cement compositions used in the bonding or fixing of implant materials, as well as the strengthening of damaged bone materials. The compositions comprise strontium-containing hydroxy apatite and a liquid component comprising bisphenol A diglycidylether dimethacrylate resin. The two components create a settable fluid substance when mixed together.

None of the references cites above disclose or suggest calcium strontium citrate or strontium substituted tri-calcium phosphate, in a formulation for oral administration, particularly when optimized by adjusting the ratio of calcium to strontium. Further, none of the prior art discloses strontium fortified calcium pharmaceutical formulations.

Coatings formed of a strontium fortified calcium hydroxyapatite are disclosed in U.S. Pat. No. 6,593,394. However, this patent does not disclose nor make obvious coatings formed of a strontium citrate or strontium substituted tri-calcium phosphate, especially optimized based on the ratio of calcium to strontium, or applied by electrophoretic deposition, which is shown to provide better properties than hydroxyapatite coatings. None of the prior art discloses nanoparticles or microparticles of strontium fortified calcium.

There exists a need for strontium fortified calcium compositions for use in implant coatings and in pharmaceutical compositions for the treatment of osteoporosis and other bone diseases and disorders.

Therefore, it is an object of the invention to provide strontium fortified calcium compositions, and methods of making and using thereof.

### SUMMARY OF THE INVENTION

Compositions containing strontium fortified calcium nanoparticles and/or microparticles, and methods of making and using thereof are described herein. The strontium fortified calcium particles contain calcium ions, calcium atoms, strontium ions, strontium atoms, and combinations thereof and one or more anions. Exemplary anions include, but are not limited to, citrate, phosphate, carbonate, and combinations thereof. Examples of preferred strontium fortified calcium compounds include, but are not limited to, calcium/strontium citrate and strontium substituted tri-calcium phosphate.

The particles can be formulated for enteral or parenteral administration by incorporating the particles into a pharmaceutically carrier. The compositions can further contain one or more pharmaceutically acceptable excipients. Suitable oral dosage forms include tablets; soft or hard, gelatin, or non-gelatin capsules; caplets, solutions, suspensions, syrups, and shakes. For parenteral administration, the microparticles are typically suspended in a pharmaceutically acceptable solvent for injection. The compositions can further contain one or more active agents useful for bone diseases or disorders, such as vitamin D, growth factors, and combinations thereof. The compositions can be used to treat or prevent one or more bone diseases or disorders, such as osteoporosis.

Alternatively, the particles can be coated onto a substrate, such as the surface of an implant. The coatings can be used to improved biocompatibility of the implant, prevent loosening of the implant, reducing leaching of metal ions from metallic implants, and reduce corrosion. The coatings can be applied to the substrate using a variety of techniques well known in the art. In one embodiment, the coating is applied using electrophoretic deposition. The use of nano- and/or microparticles that provide high surface area helps to improve interfacial strength between the coating and the implant, which allows for the use of lower sintering temperatures. Lowering sintering temperatures minimizes or prevents thermal decomposition of the coating material and/or degradation of the implant material.

### BRIEF DESCRIPTION OF THE DRAWINGS

FIG. 1 is a Fourier Transform Infrared (FTIR) spectrum of calcium and strontium citrate.

FIG. 2 is an FTIR spectrum of strontium substituted tri-calcium phosphate.

FIG. 3 is an x-ray diffraction (XRD) pattern of a strontium substituted tri-calcium phosphate mixture.

FIG. 4 is a graph showing the serum strontium levels (%) as a function of the formulation administered (control, Ca alone, Ca+L.Sr, and Ca+H.Sr). Ca+ L.Sr contained 100 mg of Ca/kg/day and 24 mg of Sr/kg/day. Ca+ H.Sr contained 100 mg of Ca/kg/day and 40 mg of Sr/kg/day.

FIG. 5 is a graph showing strontium levels in the femur and lumbar vertebra (%) as a function of the formulation administered (control, Ca alone, Ca+L.Sr, and Ca+H.Sr). Ca+L.Sr contained 100 mg of Ca/kg/day and 24 mg of Sr/kg/day. Ca+H.Sr contained 100 mg of Ca/kg/day and 40 mg of Sr/kg/day.

FIG. 6 is a graph showing the expression of various target genes: IGF-1, IGF-II, TNF-α, and Runx2 as a function of the formulation administered (control, Ca alone, Ca+L.Sr, and Ca+H.Sr). Ca+L.Sr contained 100 mg of Ca/kg/day and 24 mg of Sr/kg/day. Ca+H.Sr contained 100 mg of Ca/kg/day and 40 mg of Sr/kg/day.

FIG. 7 is a graph showing ALP activity (mol/min/mg) for various formulations at day 7, 14, and 21.

### DETAILED DESCRIPTION OF THE INVENTION

### I. Definitions

“Strontium-fortified calcium salts” and “strontium-substituted calcium compounds” are used interchangeably and refers to compounds containing calcium ions, elemental calcium, strontium ions, elemental strontium, and one or more anions. Exemplary anions include, but are not limited to, citrate, phosphate, carbonate, and combinations thereof. Examples include, but are not limited to, calcium/strontium citrate and strontium substituted tri-calcium phosphate. Mixtures of calcium compounds and strontium compounds are also within the scope of this definition.

“Nanoparticle”, as used herein, refers to particle or a structure in the nanometer (nm) range, typically from about 0.1 nm to about 1000 nm in diameter.

“Microparticle”, as used herein, generally refers to a particle of a relatively small size, but not necessarily in the micron size range; the term is used in reference to particles of sizes that can be less than 50 nm to 1000 microns or greater. In one embodiment, the diameter of the particles is from about 5 to about 100 microns, preferably from about 10 to about 50 microns, more preferably from about 10 to about 25 microns. As used herein, the microparticle encompasses microspheres, microcapsules and microparticles, unless specified otherwise. The relative sizes of microparticles and nanoparticles are such that the latter can be incorporated into the former. A micro- or nanoparticle may be of composite construction and is not necessarily a pure substance; it may be spherical or any other shape.

### II. Compositions

Nano- and microparticles of strontium fortified calcium compounds and methods of making and using thereof are described herein. The strontium fortified calcium compound contains two major components, a calcium compound or ion, a strontium compound or ion. In one embodiment, the particles are formed from a mixture of one or more calcium compounds and one or more strontium compounds. In another embodiment, the particles are formed of a calcium compound in which some of the calcium ions have been replaced by strontium ions (strontium fortified calcium compounds). The strontium fortified calcium compounds can be in the form of a pharmaceutical composition for oral administration, an implant coating, a scaffold material, or other forms, such as an injectable bone cement or filler.

Strontium fortified calcium salts can improve the bone strength caused by deteriorated osteoblast function in aging and some pathological conditions. Osteoblasts synthesize the matrix of new bone and transport calcium ions to mineralize and fill resorption cavity. Mixtures of calcium and strontium compounds or strontium fortified calcium compounds can activate the osteoblasts and provide enough calcium for bone mineralization. Due to higher osteoblast anabolic activities and higher mineralization rates, the overall density and strength of bone are improved. Calcium also has a competitive advantage over strontium in occupying calcium transporter. The affinity of transporter for calcium over strontium is only 2:1, which means that even with large amounts of calcium intake, small amounts of strontium can still be absorbed and stimulate the osteoblast activities.

A. Strontium Fortified Calcium Nano- and Microparticles

The compositions described here contain one or more strontium fortified calcium compounds, such as salts, typically in the form of nano- or microparticles. The salts contain calcium ions, calcium atoms, strontium ions, strontium atoms, and combinations thereof and one or more anions. Exemplary anions include, but are not limited to, citrate, phosphate, carbonate, and combinations thereof. Examples of preferred strontium fortified calcium compounds include, but are not limited to, calcium/strontium citrate and strontium substituted tri-calcium phosphate.

Tri-calcium phosphate is nearly insoluble in water and relies on displacement reactions in the stomach in order to dissolve the material. The incorporation of strontium in tri-calcium/strontium phosphate can improve the solubility of the compound. Tri-calcium dicitrate/strontium citrate can dissolve in water and relies less on displacement reactions. In one embodiment, the salt is a tri-calcium/strontium phosphate having a Ca:Sr:phosphate atomic ratio of 27:3:20. In another embodiment, the composition contains a mixture of calcium citrate and strontium citrate having a Ca: Sr:citrate atomic ratio of 33:6:28. The Ca:Sr ratio can be adjusted by changing the strontium and/or calcium source ratio or by further addition of one or more calcium or strontium salts. The preferred ratio of Ca:Sr is from about 9:1 to about 8:2. In one embodiment, the strontium fortified calcium compounds contains from about 1 to about 40% by moles.

Nano-size strontium phosphate particles typically have a higher surface to volume ratio and are therefore are more bioresorbable than micron size particles. By varying the ratios of nanoparticles to microparticles, the dissolution rate of the mixture can be adjusted to suit different orthopedic applications e.g., coatings that require low solubility. For bone cement fillers, the dissolution rate can be increased to facilitate bone ingrowth. In oral formulations, the mixture may contain mostly nanoparticles that have a higher dissolution rate. The strontium release rate is controlled by adjusting the calcium/strontium ratio and nanometer/micron ratio to avoid adverse toxicity.

The particles have a diameter in range from about 0.01 nm to about 1000 microns. In one embodiment, the particles have a diameter in the range from about 50 nm to about 25 microns.

B. Pharmaceutical Compositions

The strontium-fortified calcium nano- and/or microparticles can be formulated as pharmaceutical compositions. In one embodiment, the nano- and/or microparticles are formulated for oral administration. The oral dosage form can be a solid or a liquid. Suitable solid oral dosage forms include, but are not limited to, tablets; soft or hard, gelatin or non-gelatin capsules; and caplets. Suitable liquid dosage forms include, but are not limited to, solutions, suspension, and syrups.

1. Carriers, Additives, and Excipients

The pharmaceutical composition containing the nanoparticles and/or microparticles may further contain one or more pharmaceutically acceptable excipients, carriers, and additives. As used herein, the “carrier” is all components present in the pharmaceutical formulation other than the active ingredient or ingredients. The term “carrier” includes, but is not limited to, solvents, suspending agents, dispersants, buffers, pH modifying agents, isotonicity modifying agents, preservatives, antimicrobial agents, and combinations thereof.

Other additives include those useful for processing or preparation of the particles, can aid in the incorporation or stability of the strontium-fortified calcium salts, or can be useful in modifying performance of the particles.

The particles can contain other excipients including any number of other medically or pharmaceutically acceptable agents such as preservatives, lipids, fatty acids, waxes, surfactants, plasticizers, porosigens, antioxidants, bulking agents, buffering agents, chelating agents, cosolvents, water-soluble agents, insoluble agents, metal cations, anions, salts, osmotic agents, synthetic polymers, biological polymers, hydrophilic polymers, polysaccharides, sugars, hydrophobic polymers, hydrophilic block copolymers, hydrophobic block copolymers, block copolymers containing hydrophilic and hydrophobic blocks. Such excipients can be used singly or in combinations of two or more excipients when preparing microparticle compositions. These excipients can be useful in order to alter or affect drug release, water uptake, polymer degradation, stability of the bioactive agent, among other properties.

The one or more excipients can be incorporated during preparation of the of the particles. Examples of water soluble and hydrophilic excipients include poly(vinyl pyrrolidone) or PVP and copolymers containing one or more blocks of PVP along with blocks of other biocompatible polymers (for example, poly(lactide) or poly(lactide-co-glycolide) or polycaprolactone); poly(ethylene glycol) or PEG and copolymers containing blocks of PEG along with blocks of other biocompatible polymers (for example, poly(lactide) or poly(lactide-co-glycolide) or polycaprolactone); poly(ethylene oxide) or PEO, and copolymers containing one or more blocks of PEO along with blocks of other biocompatible polymers (for example, poly(lactide) or poly(lactide-co-glycolide) or polycaprolactone) as well as block copolymers containing PEO and poly(propylene oxide) or PPO such as the triblock copolymers of PEO-PPO-PEO (such as Poloxamers™, Pluronics™); and, modified copolymers of PPO and PEO containing ethylene diamine (Poloxamines™ and Tetronics™). In other aspects, the microparticle composition can be prepared containing one or more bioactive agents or one or more excipients or combinations thereof.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include) but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from OAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The proportion of pharmaceutically active neuro-enhancing agent to carrier and/or other substances may vary from about 0.5 to about 100 wt. % (weight percent). For oral use, the pharmaceutical formulation will generally contain from about 5 to about 100% by weight of the active material. For other uses, the pharmaceutical formulation will generally have from about 0.5 to about 50 wt. % of the active material.

The one or more excipients can be incorporated into the particles composition at a concentration from about 1% to about 90% by weight of the composition.

2. Controlled Release Formulations

The particles described herein can be formulated for modified or controlled release. In one embodiment, the particles can be coated with a modified or controlled release coating. Examples of controlled release coatings include extended release coatings, delayed release coatings, pulsatile release coatings, and combinations thereof. In another embodiment, the particles can be incorporated into a controlled release dosage form, such as a delayed release, extended, release, or pulsatile release dosage form.

Extended Release Dosage Forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Dosage Forms

Delayed release formulations are typically created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

3. Other Active Agents

Other active agents can be included in the compositions described herein. Suitable classes of active agents that can be co-administered with the particles described herein include, but are not limited to, vitamin D, such as vitamin D3 and/or functional equivalents thereof, growth factors, glucagon-like peptide-2, glucagon-like peptide-2 releasing compositions, non-steroidal anti-inflammatories, analgesics, and combinations thereof. In one embodiment, vitamin D, one or more growth factors, and combinations thereof are co-administered with the strontium fortified calcium particles.

### III. Methods of Making

Strontium Fortified Calcium Salts

Calcium/strontium citrate and strontium substituted tri-calcium phosphate can be synthesized using different methods. Calcium and strontium citrate mixtures can be synthesized by neutralization of a calcium and strontium hydroxide mixture with a citric acid solution at room temperature, 60° C., or boiling temperature of the reaction mixture. Due to differences in complex stability of calcium citrate and strontium citrate, separate syntheses can reduce energy input and improve the overall yield of the reaction. The optimum synthetic temperature for calcium citrate is 60° C., while strontium citrate is preferably synthesized at 100° C. As an alternative, calcium and strontium carbonate can be used for calcium and strontium citrate synthesis. Displacement reactions of strontium chloride and sodium/potassium citrate were used to produce strontium salts having a much higher (e.g., 3:2) strontium to citrate ratio.

For strontium substitute tri-calcium phosphate salt, the slow addition of the phosphoric acid allows approaching stoichiometric ratio to prevent formation of hydroxyapatite and other phosphate salts. For example, phosphoric acid is typically added at a rate of 0.01-20 ml/hour. Compared with available phosphate supplements, this high temperature synthetic method produces an anhydrous strontium fortified calcium compound with the highest Ca:phosphate and Sr:phosphate ratio. The absence of water and the presence of higher Ca:phosphate and Sr:phosphate ratios allow for higher calcium and strontium content in the final product. Adjustment of calcium, strontium and phosphate content can be achieved by adding other calcium salts or changing the Ca(OH)₂ to Sr(OH)₂ molar ratio.

In still another embodiment, strontium fortified calcium nanoparticles can be synthesized by hydrothermal treatment of diammonium hydrogen phosphate and strontium nitrate/strontium chloride precursors in the presence of a fatty acid surfactant at a temperature of 80-200° C. for a period of time ranging from about 6 to about 24 hours.

Coatings

Implant coatings are useful for preventing corrosion, enhancing biocompatibility, and increasing interfacial strength. Coatings are typically applied by spraying; however, spray techniques can suffer from a number of limitations including decomposition of the coating material and high thermal stress. Plasma spraying is typically used for coating hydroxyapatite onto implant surfaces. This method is widely used due to high interfacial strength between coating and substrate. However, the high processing cost and thermal decomposition of hydroxyapatite powder are major drawbacks of this technique.

U.S. Pat. No. 5,171,326 discloses using a microscopically powdered form of calcium-phosphate materials and electrophoretic deposition to create ceramics having significantly higher dissolution rates than previous materials. However, this method requires high temperature sintering to achieve acceptable interfacial strength.

Electrophoretic deposition can provide low cost flexible coating for hydroxyapatite on complex shape. The spraying techniques used in the prior art can result in interfacial strengths which are insufficient implant applications. Sintering of the coating has been used to improve the bonding strength between the coating and the implant. However, sintering at high temperature has two drawback, thermal decomposition of hydroxyapatite and degradation of the metal. Using nano or alternative particle that provide high surface area or chemical that can improve interfacial strength and lower down the sinter temperature makes electrophoretic deposition a viable option for implant coating.

Electrophoretic deposition of nano-particles or similar low temperature deposition technology can provide an innovative processing method with low cost and low sintering temperatures. Such methods provide an alternative to conventional plasma spraying, and are more applicable to some heat sensitive hydroxyapatite materials, such as carbonated hydroxyapatite. The preferred processing method is electrophoretic deposition, and the preferred coating material is nano-sized hydroxyapatite or calcium/strontium phosphate compound. Utilizing high surface area nano-particles such as hydroxyapatite or calcium/strontium phosphate compounds can reduce the sinter temperature to about 800° C. or less. Lower sintering temperatures minimize the decomposition and degradation problems of conventional hydroxyapatite coating. The adhesive strength test according to ASTM F1044 indicates that the bonding strength of HAp coating to a Ti substrate is 18±2.5 MPa. The cross-sectional morphology shows that the coating has a high degree of densification and no obvious pores in the coating body.

Proliferation of MSCs on nano-hydroxyapatite coating was higher than conventional hydroxyapatite or an uncoated titanium surface. The specimen was measured by XTT assay using 3′-[phenylamino-carbonyl]-3,4-tetrazolium]-bis[4-methoxy-6-nitro] benzene sulfonic acid hydrate. After four days, nano hydroxyapatite stimulated cell growth on the surface of coating and also in the region around the materials. Based upon the XTT assay, MSCs proliferate fastest on nano-strontium fortified calcium particles and slowest on the Ti sheets.

Nano-strontium fortified calcium particle coatings were produced by electrophoretic deposition (EPD) method and sintered at temperatures from 20-1200° C., preferable lower than 800° C. This method decreases the thermal stress of the coating and the cost of production is lower than plasma spraying methods. Utilization of strontium containing nano-hydroxyapatite and special treatment method eliminate the problem of crack development.

Suitable substrates include, but are not limited to metallic and ceramic substrates. Examples of metallic substrates include, but are not limited to, tantalum, cobalt, chromium, cobalt alloys, chromium alloys, titanium, titanium alloys, ceramics, and combinations thereof.

### IV. Method of Using Strontium Fortified Calcium Salts

The compositions described herein can be used for the treatment and/or prevention of bone diseases or disorders, such as osteoporosis. The compositions are typically administered as a solid or liquid oral dosage formulation. Alternatively, the compositions described herein can be used as an injectable bone filler or scaffold to treat or prevent bone fractures or other related bone diseases.

Pharmaceutical Compositions

In osteoporosis patients, stand alone calcium supplement treatment does not significantly improve the bone mineral density due to impair osteoblast function. Without osteoblast synthesizing new matrix material, no new bone will form despite adequate serum calcium concentration. High serum calcium concentration suppresses resorption, which explains why calcium supplements can reduce bone loss in osteoporosis patient. Strontium fortified calcium salts can improve the bone strength caused by deteriorated osteoblast function in aging and some pathological condition. Osteoblast synthesizes the matrix of the new bone and transports calcium ion to mineralize and fill resorption cavity. Strontium combined with calcium salt can activate the osteoblast and provide enough calcium for bone mineralization. Due to higher osteoblast anabolic activities and higher mineralization rate the bone overall density and strength are improved. Calcium has competitive advantage in occupying calcium transporter against strontium. The affinity of transporter to calcium: strontium is only 2:1. Even with large amount of calcium intake small amount of strontium can still be absorbed and stimulate the osteoblast activities. By varying the Ca:Sr ratio, one can alter the strontium absorption rate in the body.

Based on animal studies described herein, strontium fortified calcium salt has shown significant improvement in osteogenic factors expression and bone mineral density compared with control and standalone calcium salt treatment groups. Combined treatment (low dose Sr) increased bone volume, primarily by increasing trabecular thickness (see the examples). Ca treatment alone decreased the mineral apposition rate, while calcium plus strontium treatment increased the mineral apposition rate (see the examples).

Evaluation of the mechanical properties of a single trabecula of lumbar vertebra showed that while there was little or no change in the elastic modulus and only a slight decrease in hardness, the increased E/H proportional to fracture toughness) resulted in the fracture risk reduction observed after strontium treatment.

The examples also show that although mRNA expression levels of IGF-II showed no significant difference in all four groups in the study, IGF-I gene transcript level was Sr dose dependently up regulated. IGF-I gene expression was increased and significantly up regulated in animals receiving strontium fortified calcium compositions when compared to animals receiving Ca administration alone. Similarly, administration of the strontium fortified calcium compositions resulted in an increase in the expression of Runx2 mRNA while treatment in G2 slightly decreased the Runx2 expression. On the other hand, levels of mRNA gene transcripts for TNF-α were significantly decreased in animals receiving calcium alone or in combination with strontium as compared to a control. These findings indicate that strontium plus calcium can stimulate osteogenic gene expression and thus induce new bone formation.

The dosage range for oral administration is typically less than about 100 mg/kg/day, preferably from about 24-40 mg/kg/day.

Coatings

For implant fixation, implant surface treatment plays an important role in preventing interface dislocation. Stable, effective coatings can prevent the loosening of the implant, reduce the leaching out of metal ions from the implant, and reduce corrosion of the implant, particularly metallic implants. Coatings prepared from high surface area strontium fortified calcium particles enhance biocompatibility, increase interfacial strength, decrease sinter temperature, and/or reduce thermal stress. The coatings are typically prepared from particles having a diameter from about 10 nm to about 250 microns.

In one embodiment, the coating material promotes differentiation of mesenchymal stem cells (MSCs) and/or proliferation of osteoblasts and/or improves bone densification. The coating can further contain one or more active agents, such as agents that promote bone regeneration. Suitable classes of active agents that can be co-administered with the particles described herein include, but are not limited to, vitamin D, such as vitamin D3 and/or functional equivalents thereof, growth factors, glucagon-like peptide-2, glucagon-like peptide-2 releasing compositions, non-steroidal anti-inflammatories, analgesics, and combinations thereof. In one embodiment, vitamin D, one or more growth factors, and combinations thereof are co-administered with the strontium fortified calcium particles.

Scaffolds

The strontium fortified calcium particles can be used to prepare scaffold for bone tissue engineering. Methods for preparing such scaffolds are well known in the art. In one embodiment, the strontium fortified calcium particles are mixed with chitosan-gelatin, alginate, and/or collagen by phase separation to form the scaffolds.

Bone Fillers

In one embodiment, the strontium fortified particles can be used to prepare a bioactive bone filler or cement. For example, the particles can be mixed with a binder, such as calcium sulfate, to form the filler or cement. Bone cements can be used to fill a void or affix bone and/or orthopaedic hardware to bone. Bone fillers are typically used to fill voids or defects in bone. Methods for preparing and administering bone fillers and/or cements are well known in the art. The fillers and/or cements can be formulated to provide a sustained release of the strontium fortified calcium compounds. As discussed above, the strontium fortified calcium compounds have been shown to increase osteogenic factors expression and bone mineral density compared with control and standalone calcium salt treatment groups and can stimulate osteogenic gene expression and thus induce new bone formation. Strontium fortified calcium bone fillers can be applied to stabilize the bone plate or screw, or restore stability of orthopedic implants which have loosened.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Synthesis of Strontium Citrate and Preparation of Strontium Citrate/Calcium Citrate Blends

### Synthesis of Strontium Citrate

0.5 moles of citric acid monohydrate was dissolved in 500 ml of distilled water. 0.5 moles of strontium hydroxide was added to the citric acid solution in a few batches with stirring and heating. The reactor was covered with glass to prevent spillage of the viscous liquid. The solution was degassed using ultrasound to help reduce the carbonate content of the final product. For general food grade or supplement usage, 1-2% carbonate is acceptable. For a higher purity product, the reactor was filled with an inert gas, such as argon or nitrogen.

After heating the solution for 1 hour, the solution was cooled to room temperature. Addition of an organic solvent such as acetone, ethanol or propanol to stabilize the complex further improved the yield of the reaction. The precipitate was dried at 100° C. The strontium citrate salt was mixed with calcium citrate by mechanical means. The reactions describing the synthesis of strontium citrate and calcium citrate are shown below:

Sr(OH)₂+C₆H₈O₇→Sr(C₆H₆O₇)+2H₂O

3Ca(OH)₂+2C₆H₈O₇→Ca₃(C₆H₅O₇)₂+3H₂O

The strontium citrate/calcium citrate blend was prepared by roughly mixing strontium citrate and calcium citrate in a ceramic bowl. The mixture was sieved repeatedly until a homogenous mixture was obtained. This method eliminates bulk particles and produces a homogenous mixture. The size of the strontium fortified calcium citrate particles was from 2-16 microns.

### Example 2

### Synthesis of Strontium Substituted Tri-calcium Phosphate

3.67 moles of Ca(OH)₂ and 0.41 moles of Sr(OH)₂.8H₂O were dissolved in 8 L distilled water and stirred for one hour. 2.72 moles (182.4 ml) of H₃PO₄ was added dropwise at a rate of 2 ml/hour via a syringe pump. After the addition of H₃PO₄, the solution was stirred for one to two days. The tri-calcium/strontium phosphate precipitate was filtered using vacuum suction and dried in an oven at 110° C. for 12 hours. The precipitate was milled and baked in the oven again for 6 hours. The precipitate was then heat treated in a furnace at 800° C. for three hours. The temperature of the furnace was raised 50° C./15 minutes until it reached 800° C. After heating at 800° C. for three hours, the furnace was cooled, and a greenish white powder was obtained. The yield was approximately 84.8%. The particle size of the strontium substituted tricalcium phosphate was 100 nm-7.41 μm. Without heat treatment, the particle size was 100 nn-24.14 μm.

Reaction of Ca(OH)₂, Sr(OH)₂ and H₃PO₄ give intermediate deficient hydroxyapatite:

9Ca(OH)₂+Sr(OH)₂+6H₃PO₄→Ca₉₋ₓSr₁₋ₓ(HPO₄)ₓ(PO4)₆₋ₓ(OH)₂₋ₓ.

The deficient apatite converted to β-strontium substituted calcium phosphate

Ca₉₋ₓSr₁₋ₓ(HPO₄)ₓ(PO4)₆₋ₓ(OH)₂₋ₓ→3Ca_{2.7}Sr_{0,3}(PO₄)₂+H₂O.

To reduce particle agglomeration, a small amount of n-butanol was added to the final mixture during the filtration process.

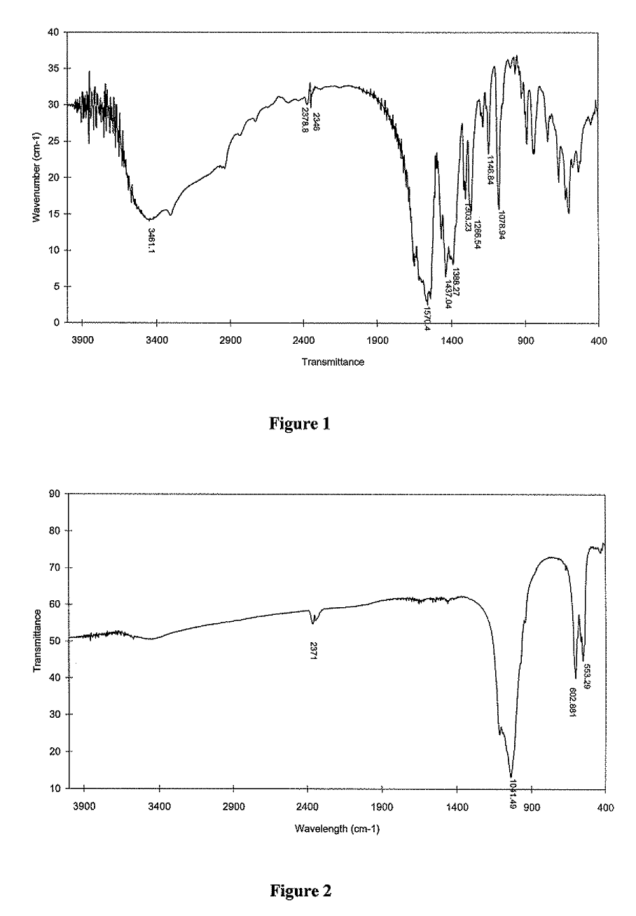

For final product analysis, inductively couple plasma-atomic emission spectroscopy (ICP-ABS) was used due to lack of strontium (Sr) interference. ICP-AES was used to obtain the exact calcium and strontium content of the strontium fortified calcium compound. Scanning Electron Microscope/Energy Dispersive Using X-Ray (SEM/EDX) analysis was used to determine particle size and Ca/Sr homogeneity in the sample. FTIR results are shown in FIGS. 1 and 2. The spectra were compared with standard spectra.

FIG. 1 is an IR spectrum of calcium/strontium citrate. FIG. 1 shows that anti-symmetric and symmetric mode band attributed to —COO⁻ was present at 1550-1650 cm⁻¹ & 1440-1360 cm⁻¹ respectively. Strong and wide absorptions attributed to —OH with hydrogen bond band at 3000-3600 cm⁻¹ showed the mixture was a hydrated salt.

FIG. 2 is an FTIR spectrum of strontium substituted tri-calcium phosphate. The IR spectrum indicates the existence of tri-strontium phosphate and β-TCP in the compound. Absorption at 604 cm⁻¹ and 1033 cm⁻¹ showed the presence of PO₄ in the sample.

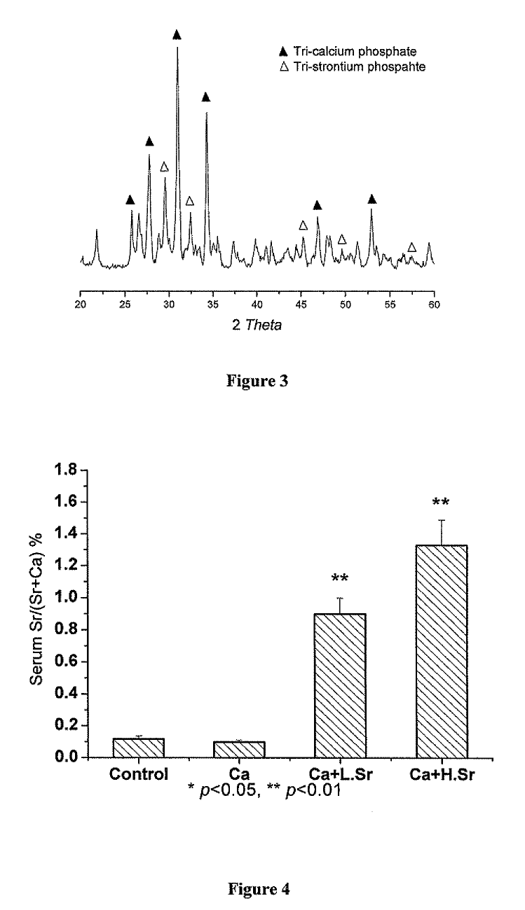

X-ray diffraction (XRD) pattern of tri-calcium/strontium phosphate is shown in FIG. 3. The peaks pattern is the combination of β-tricalcium phosphate and tri-strontium phosphate.

### Example 3

### Synthesis of Nano-Calcium/Strontium Citrate

A micro-emulsion was prepared by placing 0.1M sodium citrate (solution A, 280 mL) and a mixture of 0.1M calcium chloride and 0.05M strontium chloride (solution B, 450 mL) in a dispersing apparatus. The mixture was homogenized at 5000 rpm for 45 minutes. The solid phase was separated out by centrifugation. The precipitate was washed with ethanol/dichloroethane and further washed with ethanol three times. The nano-particles were dried at 100° C. The resulting particles had a length of about 500 nm and a thickness from about 35-150 nm.

### Example 4

### Coating of Nano-Calcium and Strontium Material on Implant

### Synthesis of Strontium Fortified Calcium Nano-Particles

Solutions of Ca(NO₃)₂, Sr(NO₃)₂ and (NH₄)₂HPO₄ were adjusted to a pH greater than 10 with ammonia. The solutions were mixed in a stoichiometric ratio of Ca+Sr to P=1.67. After the reaction was finished, the precipitate was rinsed several times with distilled water until the pH value decreased to 7. In order to avoid serious aggregation of the ultra-fine powder during drying, the water in the precipitate was replaced by n-butanol. The precipitate was then dried at 80° C. and calcined at 400° C. to remove any organic residues. Trace agglomerate particles were removed by sieving before deposition.

Preparation of Ti Surface

Titanium was cut into the desired size and polished with silicon carbide paper. Surface roughness treatment was conducted for titanium followed by etching and oxidation using a 1:1 sulfuric acid and hydrogen peroxide mixture. A nano-strontium fortified calcium particle coating was prepared through EPD process (10V DC field, 1 min) and sintered at 800° C.

### Example 5

### In Vivo Studies of Strontium-Fortified Calcium Compositions

18 mountain goats, aged 6-8 years, were ovariectomized to induce osteoporosis 1 year before the beginning of the commencement of treatment. The animals were given a random number to obscure identification of histomorphometric samples by observers. The goats were randomly divided into four groups as shown in Table 1.

TABLE 1
Animal Studies

Treatment

| Group | mg Ca/kg/day | mg Sr/kg/day | Number |
|---|---|---|---|
| Control (G1) | 0 | 0 | 3 |
| Ca (G2) | 100 | 0 | 5 |
| Ca + L. Sr (G3) | 100 | 24 | 5 |
| Ca + H. Sr (G4) | 100 | 40 | 5 |

Ca + L. Sr contained 100 mg of Ca/kg/day and 24 mg of Sr/kg/day.
Ca + H. Sr contained 100 mg of Ca/kg/day and 40 mg of Sr/kg/day.

Fourteen days and 3 days before sacrifice, each animal was injected with 20 mg/kg of tetracycline (Terramycin, Pfizer) to obtain a double fluorescent label at the sites of active bone formation. All animals were sacrificed 16 weeks after the onset of treatment.

Serum Strontium Levels

Serum calcium and strontium levels were measured before and after treatment. The measurements are shown in Table 2.

TABLE 2
Serum strontium levels before and after treatment

| | Pre-treatment | | Post-treatment | |
|---|---|---|---|---|
| Groups | Ca (mg/L) | Sr (mg/L) | Ca (mg/L) | Sr (mg/L) |
| G1 | 96.8 ± 5.657 | 0.240 ± 0.014 | 112.83 ± | 0.293 ± 0.101 |
| | | | 19.300 | |
| G2 | 94.9 ± 5.726 | 0.285 ± 0.035 | 101.40 ± | 0.214 ± 0.027 |
| | | | 15.365 | |
| G3 | 94.4 ± 1.131 | 0.315 ± 0.007 | 90.40 ± | 1.698 ± 0.303 |
| | | | 16.199 | |
| G4 | 92.8 ± 3.124 | 0.265 ± 0.035 | 109.06 ± | 3.092 ± 0.691 |
| | | | 20.541 | |

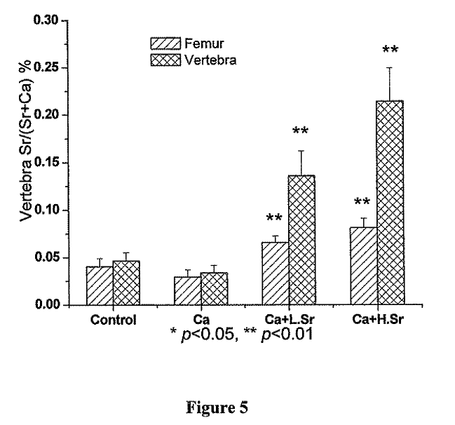

The serum strontium levels after treatment are shown graphically in FIG. 4. In the group that received Ca treatment only (G2), the levels of serum Sr concentration did not differ from those in control group. Mean levels of Sr concentrations in serum were 0.29±0.09 and 0.21±0.03 mg/L in G1 and G2 group, respectively. In contrast, Sr concentrations in serum increased considerably to 1.70±0.30 and 2.10±10.70 mg/L in G3 and G4, respectively. Sr mole ratio increased dose-dependently with treatment (0.9%, 1.3% Sr/(Sr+Ca) with G3 and G4, respectively.

Bone Strontium Levels

Strontium levels in the femur and lumbar vertebra are shown in FIG. 5. Bone Sr levels in the lumbar vertebra increased dose-dependently in Sr treated groups by four- and six-fold in G3 and G4, respectively. Furthermore, Sr levels in the femur increased to 571±158 and 738±42 mg/kg in G3 and G4, respectively.

Bone Density

The effects of Ca alone or in combination with Sr treatments on lumbar vertebra adjusted bone mineral density (BMD) differed between the groups. The results are shown in Table 3.

TABLE 3
Micro-architecture of lumbar vertebra as determined by micro
computed tomography (micro CT)

| | Experimental Groups | | | |
|---|---|---|---|---|
| Parameter | Control | Ca | Ca + L. Sr | Ca + H. Sr |
| BMD | 191.606 ± 17.383 | 198.365 ± 10.123 | 208.074 ± 10.326* | 201.927 ± 1.169 |
| Apparent | | | | |
| BMD | 765.760 ± 18.401 | 749.037 ± 21.540 | 752.585 ± 31.691 | 743.337 ± 44.581 |
| Material | | | | |
| BV/TV | 0.180 ± 0.006 | 0.184 ± 0.009 | 0.202 ± 0.013* | 0.181 ± 0.017 |
| Tb. Th* | 0.164 ± 0.017 | 0.186 ± 0.020 | 0.188 ± 0.001* | 0.190 ± 0.016* |
| (mm) | | | | |
| Tb. Sp* | 0.474 ± 0.062 | 0.519 ± 0.135 | 0.435 ± 0.048 | 0.432 ± 0.045 |
| (mm) | | | | |
| Tb. N* | 2.337 ± 0.593 | 2.123 ± 0.656 | 2.559 ± 0.432 | 2.618 ± 0.530 |
| (1/mm) | | | | |
| SMI | 0.491 ± 0.021 | 0.427 ± 0.073 | 0.485 ± 0.110 | 0.498 ± 0.058 |
| Conn. D | 2.955 ± 0.784 | 3.419 ± 0.036 | 3.789 ± 1.010 | 4.003 ± 2.008* |
| (1/mm³) | | | | |

Combined treatment (low dose Sr) increased bone volume, primarily by increasing trabecular thickness (see the measurements in bold in Table 3). The BMD of the lumbar vertebra increased by 9.4% in G4.

The bone mineral apposition rate (MAR) of the four groups were 1.95±0.23, 1.65±0.20, 2.03±0.25 and 2.10±0.22 μm/day, respectively. Ca treatment alone (G2) decreased the mineral apposition rate by 15.4%, while Ca+L.Sr treatment increased the mineral apposition rate by 4.1%. Ca+H.Sr treatment, however, increased the mineral apposition rate by 7.9% (p=0.026).

Mechanical Studies

Nanoindentation Studies

Indentation tests, sometimes called hardness tests, are perhaps the most commonly applied means of testing the mechanical properties of materials. The technique has its origins in the Mohs scale of mineral hardness, in which materials are ranked according to what they can scratch and are, in turn, scratched by. The characterization of solids in this way takes place on an essentially discrete scale, so much effort has been expended in order to develop techniques for evaluating material hardness over a continuous range. More recently (ca. 1975), the nanoindentation technique has been established as the primary tool for investigating the hardness of small volumes of material.

In nanoindentation, small loads and tip sizes are used, so the indentation area may only be a few square micrometres or even nanometres. This presents problems in determining the hardness, as the contact area is not easily found. Atomic force microscopy or scanning electron microscopy techniques may be utilized to image the indentation, but can be quite cumbersome. Instead, an indenter with a geometry known to high precision (usually a Berkovich tip, which has a three-sided pyramid geometry) is employed. During the course of the instrumented indentation process, a record of the depth of penetration is made, and then the area of the indent is determined using the known geometry of the indentation tip. While indenting, various parameters, such as load and depth of penetration, can be measured. A record of these values can be plotted on a graph to create a load-displacement curve. These curves can be used to extract mechanical properties of the material.

Nanoindentation tests were performed on a single trabecula of lumbar vertebra. The results are shown in Table 4.

TABLE 4
Mechanical properties of a single trabecula of a lumbar vertebra
after treatment

| | | | |
|---|---|---|---|
| | Elasitc Modulus, | Hardness, H | |
| Group | E (Gpa) | (MPa) | E/H |

| | | | |
|---|---|---|---|
| Control | 14.025 ± 0.017 | 685.800 ± 37.937 | 20.790 ± 0.707 |
| Ca | 12.652 ± 1.102 | 610.146 ± 62.170 | 20.970 ± 0.461 |
| Ca + L. Sr | 14.017 ± 0.297 | 639.959 ± 63.869 | 22.378 ± 2.100 |
| Ca + H. Sr | 13.542 ± 0.665 | 642.025 ± 28.620 | 22.187 ± 0.720 |

As shown in Table 4, there was little or no change in the elastic modulus and only a slight decrease in hardness. However, the increased E/H proportional to fracture toughness) is one of the reasons for the fracture risk reduction observed after strontium treatment.

Compression on the whole vertebral body was also measured. The results are shown in Table 5.

TABLE 5
Maximum load and stiffness

| Group | Maximum Load (N) | Stiffness (N/mm) |
|---|---|---|
| Control | 5292.50 ± 900.15 | 3085.24 ± 554.21 |
| Ca | 5219.00 ± 1598.45 | 2906.69 ± 544.24 |
| Ca + L. Sr | 6467.00 ± 959.11 | 2975.23 ± 278.21 |
| Ca + H. Sr | 5518.50 ± 849.23 | 3233.85 ± 370.31 |

Expression of Target Genes

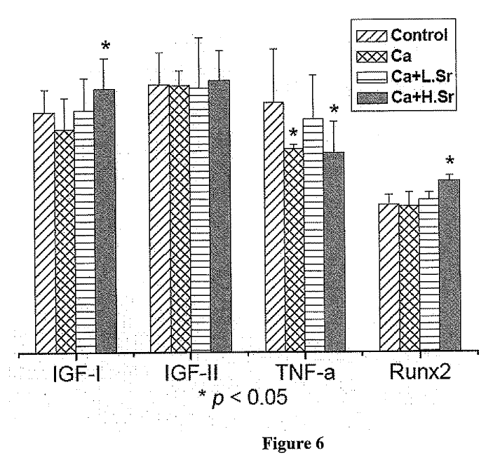

Bone matrix levels of IGF-I, IGF-II, TNF-α, and Runx2 were determined as markers of bone remodeling. The results are shown in FIG. 6. Although mRNA expression levels of IGF-II showed no significant difference in all four groups in this study, IGF-I gene transcript level was Sr dose dependently up regulated. IGF-I gene expression was increased in G3 and significantly up regulated in G4 animals when compared to G1 animals receiving Ca administration alone.

Similarly, administration of the formulation to G3 and G4 resulted in an increase in the expression of Runx2 mRNA while treatment in G2 slightly decreased the Runx2 expression. On the other hand, levels of mRNA gene transcripts for TNF-α were significantly decreased in G2, G3 and G4 when compared to G1. These findings indicate that strontium plus calcium can stimulate osteogenic gene expression and thus induce new bone formation.

Osteoblast Differentiation

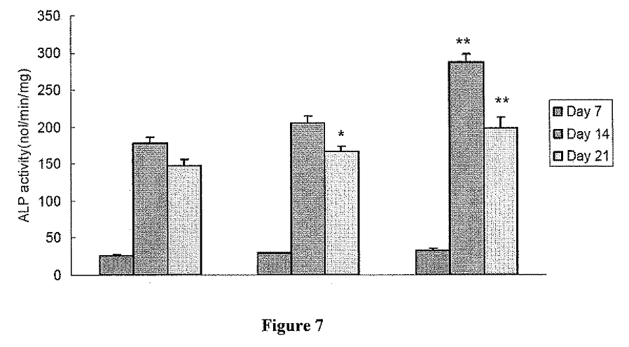

It was also observed that the administration of strontium promoted osteoblast differentiation. This was confirmed via alkaline phosphatase (ALP) staining and ALP quantification. The results of the ALP quantification are shown in FIG. 7. As shown in FIG. 7 and confirmed via ALP staining, strontium treatment increased osteoblast differentiation particularly after 14 days. Strontium treatment also increased matrix mineralization of osteoprogenitors.

### Example 6

### Preparation of Strontium Hydroxyapatite Nanowires

5 g of sodium hydroxide, 40 g of oleic acid and 160 ml of ethanol were mixed under agitation. The strontium nitrate solution was prepared by dissolving 6.44 g strontium nitrate in 75 ml distilled water. The strontium nitrate solution was added to the above mixture. 5 g of sodium phosphate was dissolved in 75 ml distilled water. The sodium phosphate solution was added to the strontium oleic acid salt mixture. The mixture was agitated for about 10 minutes and then transferred to a 500 ml Teflon lined autoclave, which was sealed and hydrothermally treated at 160° C. for 15 hours.

Since fatty acid strontium salts have a higher density than their calcium counterpart, washing with distilled water is essential to remove the fatty acid strontium salt, followed by washing with 70% ethanol and dehydrated absolute ethanol.

The nanowires were characterized using Scanning Electron Microscopy (SEM). The length of the nanowires was about 2.43±0.6 μm and diameter was about 81±12 nm.

We claim:

1. A pharmaceutical composition comprising nanoparticles, microparticles, or combinations thereof comprising one or more strontium-fortified calcium compounds, wherein the one or more strontium-fortified calcium compounds comprise calcium ions and strontium ions and one or more anions selected from the group consisting of phosphate, citrate, carbonate, and combinations thereof.

2. The composition of claim 1 formulated as an oral dosage form selected from the group consisting of tablets, capsules, caplets, solutions, syrups, and suspensions.

3. The composition of claim 1, wherein the composition in the form of a coating on an implant.

4. The composition of claim 1, wherein the composition is in the form of a bone cement or filler.

5. The composition of claim 4, wherein the composition further comprises a binder.

6. The composition of claim 5, wherein the binder is calcium sulfate.

7. The composition of claim 1, wherein the particles are in the form of a nanowire.

8. The composition of claim 1, wherein the nanoparticles have a diameter of at least about 10 nm and the microparticles have a diameter of between about 10 and about 250 microns.

9. A method for treating a bone disease or disorder, the method comprising administering an effective amount of a composition comprising nanoparticles, microparticles, or combinations thereof comprising one or more strontium-fortified calcium compounds, wherein the one or more strontium-fortified calcium compounds comprise calcium ions and strontium ions and one or more anions selected from the group consisting of phosphate, citrate, carbonate, and combinations thereof.

10. The method of claim 9, wherein the bone disease or disorder is osteoporosis.

11. The method of claim 9 in the form of an oral dosage form selected from the group consisting of tablets, capsules, caplets, solutions, syrups, and suspensions.

12. The method of claim 9, wherein the composition in the form of coating on an implant.

13. The method of claim 9, wherein the composition is in the form of a bone cement or filler.

14. The method of claim 13, wherein the composition further comprises a binder.

15. The method of claim 13, wherein the binder is calcium sulfate.

16. The method of claim 8, wherein the nanoparticles have a diameter of at least about 10 nm and the microparticles have a diameter of between about 10 and about 250 microns.

17. A method for coating strontium fortified calcium nanoparticles, microparticles, and combinations thereof onto a substrate, the method comprising electrophoretically depositing a high surface area nanoparticles, microparticles, and combinations thereof comprising one or more strontium-fortified calcium compounds, wherein the one or more strontium-fortified calcium compounds comprise calcium ions and strontium ions and one or more anions selected from the group consisting of phosphate, citrate, carbonate, and combinations thereof, wherein the deposited material is sintered at a temperatures less than 800° C.

18. The method of claim 17, wherein the size of the particles is from about 10 nm to about 250 μm.

19. The method of claim 17, wherein the substrate is selected from the group consisting of tantalum, cobalt, chromium, cobalt alloys, chromium alloys, titanium, titanium alloys, ceramics, and combinations thereof.

20. The method of claim 17, wherein the coating material further comprises a material that promotes bone-integration.

21. The method of claim 17, wherein the coating material promotes differentiation of mesenchymal stem cells or proliferation of osteoblasts.

22. The method of claim 16, wherein the coating is porous or non-porous.
